(19) United States
(12) Patent Application Publication
(10) Pub. No.: US 2011/0092872 A1
(43) Pub. Date: Apr. 21, 2011

(54) MEDICAL COMPRESSION THERAPY MATERIAL, BANDAGE AND CLOTHING PIECE

(76) Inventor: Hermann CHRISTIANSEN, Wettstetten (DE)

(21) Appl. No.: 12/907,055

(22) Filed: Oct. 19, 2010

(30) Foreign Application Priority Data
Oct. 21, 2009 (DE) 10 2009 050 031.6

(51) Int. Cl. A61F 13/00; B32B 3/02; A41B 11/00; A41C 3/00; A61F 13/08

(52) U.S. Cl. 602/53; 428/114; 2/239; 450/93

(57) ABSTRACT

A material for a product for the compression of body parts includes a two-dimensional base body. At least one linear protuberance, which extends longitudinally in a basic direction, is formed on at least one flat side of this base body. The at least one protuberance has at least one section which is aligned obliquely and/or transversely to its basic direction.

### BACKGROUND OF THE INVENTION

The invention relates to a material for a product for the compression of body parts, as well as to a bandage and pieces of clothing made from this material.

Materials for the compression of body parts are used, for example, with medical compression therapy, in order to relieve a disturbed or damaged vein system and/or lymph system. Here, a pressure is exerted onto the body part to be treated, by a bandage or a piece of clothing which is specially adapted for this, for example by a compression stocking, in order to prevent the occurrence of edemas, or in order to reduce the size of existing edemas by drainage.

It is usual to provide the material of the applied bandages and pieces of clothing with protuberances at its side which comes into contact with the body part, for partial pressure increase onto the body part, in order to improve the effectiveness of these bandages and pieces of clothing. In this context, such protuberances are known, which are designed in a nap-like manner and such protuberances which have the shape of straight lines.

### BRIEF SUMMARY OF THE INVENTION

Against the background of this prior art, it is an object of the invention to provide a material for a product for the compression of body parts, with which material one may achieve improved drainage effects compared to the materials which have been known until now. A further object of the invention lies in the creation of a bandage and the creation of pieces of clothing from these materials.

This object is achieved by a material for a product for the compression of body parts or a material stuck onto the skin, wherein the material comprises a two-dimensional base body, wherein at least one linear protuberance, which extends longitudinally in basic direction, is formed on at least one flat side, wherein the protuberance comprises sections which are aligned obliquely and/or transversely to their basic direction, and by a bandage and by a piece of clothing formed from this material. Advantageous further embodiments of the material, the bandage as well as the piece of clothing, are to be deduced from the claims, the subsequent description, as well as the drawings. Here, according to the invention, the features described, on their own, but also in combination, may further form the inventive solutions of the material, the bandage or the clothing.

The material according to the invention, for a product for the compression of body parts, which in particular is envisaged for medical compression therapy, comprises a two-dimensional base body. At least one linear protuberance and preferably a multitude of such protuberances, which extend longitudinally in a basic direction, are formed on at least one flat side of this base body, this flat side being typically envisaged for bearing on the body part. Within the context of the invention, a product for the compression of body parts is not only to be understood as a product for medical compression therapy, but also each and any product which comes to bear on the body parts and thus exerts a certain pressure onto these body parts. These, for example, may also be pieces of clothing which come to bear in a tightly fitting manner or the like, in particular even sports clothing

According to the invention, the at least one protuberance or the protuberances in each case have sections which are aligned obliquely and/or transversely to their basic direction. This means that at least one protuberance is not designed linearly in a straight manner, but instead, despite a clearly defined basic direction, it has a curved course with indents transverse to the basic direction. The indents, which in each case have a common direction transversely to the basic direction or may be aligned transversely to the basic direction in both opposite directions, may be designed in an angular as well as rounded manner. The at least one protuberance, by being designed in a curved manner, has a larger length compared to a straight-line protuberance, with respect to an equally large base body. Accordingly, the surface exerting a pressure on the body tissue undergoing therapy, with the material according to the invention, is larger compared to the materials used until now, which leads to an increased drainage capability. A further advantage of the material according to the invention is to be seen in the fact that the tissue fluid displaced by the at least one protuberance is not displaced in only one preferred direction, which is to say transversely to the longitudinal direction, as with the straight-lined protuberances which are common until now, but is led in several different directions on account of the regions directed obliquely or transversely to the basic direction of the protuberance, and is thus discharged in a better distributed manner.

As has already been noted, it is conceivable to design the course of the at least one protuberance in the form of a zigzag line or in a differently angular manner. Preferably, the at least one protuberance however is meandering in the basic direction. Accordingly, the contour of the at least one protuberance in plan view has the shape of a serpentine line with rounded loops which extend in both opposite directions transversely to the basic direction of the protuberance. The degree of curvature of these loops basically may be freely selected in adaptation to the respective drainage task.

Usefully, several protuberances are formed next to one another in their basic direction, on the base body of the material according to the invention. Here, the basic directions of the individual protuberances may indeed differ, wherein however it is advantageous if adjacent protuberances do not intersect, so that the intermediate space between adjacent protuberances forms an unhindered discharge region for tissue fluid which is to be led away. Preferable is a design in which adjacent protuberances run essentially parallel to one another, thus in each case have a common basic direction.

The cross-sectional contour of the protuberances is basically likewise infinite. Preferred however is a design in which the protuberances form a preferably sinusoidal curvature which is directed normally to the flat side of the base body. The height of these curvatures, which is to say the distance of the apex of the curvatures to the base body, just as the maximal width of the curvatures, may be up to 30 mm, depending on the drainage task.

Further preferably, adjacent protuberances are arranged spaced from one another. This is advantageous inasmuch as an intermediate space is thus created between the protuberances, in which the tissue fluid displaced by the protuberances may flow away without external pressure application or with at least a significantly lower external pressure application. The distance between adjacent protuberances may preferably be up to 30 mm, wherein the finally selected distance is to be selected depending on the drainage task to be fulfilled. Optionally, adjacent protuberances on a common base body may also have different distances from one another.

Moreover, it may be advantageous if adjacent protuberances border one another in an essentially direct manner, as is envisaged in a further preferred design of the material according to the invention. This means that the protuberances may be arranged quasi directly next to one another or at a negligible distance to one another. By this, one may achieve a particularly large surface pressure on the tissue to be retreated.

Advantageously, a textile may form the base body of the material according to the invention. Accordingly, a bandage of natural and/or plastic fibers, in the form of a cloth for example, may form the base body. The use of elastic plastic fibers may here give the base body the elasticity which, as a rule, is required with products for medical compression therapy.

Apart from this, it may also be advantageous if the base body is formed by a film or a foam body. Thus, for example, preferably gas-impermeable plastic films may form at least a part of an inflatable compression sleeve which is envisaged for bearing on a body part. The use of a foam body as a base body of the material according to the invention, for example, has the advantage that such a foam body may be very simply manufactured and, optionally, may also be processed further in a simple manner.

The at least one protuberance may advantageously be deposited on the base body. Thus, the protuberance may, for example, be injected or bonded on the surface of the base body, this surface being smooth as seen macroscopically, and preferably with textile base bodies may also be stitched on the base body.

Apart from this, the at least one protuberance may also advantageously form a part of the base body, thus be an integral component of the base body. Such a design in particular lends itself if a plastic film or a foam body forms the base body, since the protuberance or the protuberances with such base bodies may be formed out on this base body in a simple manner when manufacturing the base body, which is to say the plastic film or the foam body.

The invention also relates to all types of products for the compression of body parts of a material according to the preceding description. Thus, the invention also relates to a bandage. Bandages in the context of the invention are to be understood as all means with which a body part may be covered in a large-surfaced manner in order, for example, to protect it from harmful environmental influences or mechanical loading, or which serve for compression, hemostasis, secretion, absorption, or occlusion. Accordingly, these may preferably be medical bandages, for example in the form of swathes, dressings and wound coverings, or even bandages which are used in the field of sports for preventing injuries.

The bandage according to the invention, in particular a medical bandage, at least in sections and preferably in a region which is envisaged for directly bearing on a body part, is formed from the material according to the invention, which has at least one of the features described above. A bandage may be designed as a dressing which is to be wound around a body part or also as a hose-like sleeve. The basic dimensions, which is to say length and width of a bandage dressing, are to be infinitely selected. The dimensions of a bandage designed as a sleeve are typically to be adapted to the size and shape of the body part on which it is to be applied.

With the bandage according to the invention, the basic direction of the at least one protuberance preferably extends transversely to the longitudinal extension of the bandage, wherein longitudinal extension of a bandage designed as a hose-like sleeve is to be understood as its peripheral direction. The alignment of the protuberance or protuberances on the bandage is thus preferably of the type such that the protuberance or protuberances and free spaces formed therebetween extend in the longitudinal direction of the leg, if the bandage is applied onto a leg for example. This is advantageous inasmuch as tissue fluid, which is displaced by the protuberances into the tissue lying between the protuberances, may flow away in an uninhibited manner and, as the case may be, supported by gravity.

The bandage may advantageously be designed as an adhesive bandage. Thus, for example, a bandage designed as a swathe may be designed as an adhesive dressing. A glue or adhesive means may be deposited on at least one section of a flat side of a bandage or dressing. Here, one preferably envisages the glue being deposited on that flat side of the bandage or dressing, which is provided with the inventive protuberances. It is thus possible to deposit the adhesive directly onto the protuberances or to use the intermediate spaces between adjacent protuberances as a depot for the adhesive. Typically, it is also possible to provide the protuberances as well as also the regions between the protuberances with an adhesive layer. The adhesive means may also be deposited onto the bandage or onto the dressing in the form of protuberances, so that the adhesive forms at least a part of the protuberance.

The invention further relates to a piece of clothing which, at least in sections, is formed of a material with at least one of the characteristics described above. Such a piece of clothing may form a product for the compression of body parts, at least in individual regions. Advantageously, the piece of clothing may be a stocking Such stockings are known as compression stockings or support stockings and, for example, serve for retaining a deblockage condition in a leg. Further preferably, the piece of clothing according to the invention may also be a bra which may be applied, for example, after a breast cancer operation, which spares the breast. Complications, as for example a lymphatic edema or a fibrosis, may occur after such an operation. Here, manual lymph drainage may lead to a relief, wherein the bra according to the invention may be applied for maintaining this condition, and this bra is provided with the material according to the invention, at least in the cup which receives the breast concerned.

Moreover, one may also manufacture any other piece of clothing from the material according to the invention. Thus, for example, it is conceivable to use this material also with pantyhose, corsages, swimwear, underwear, sports clothing, slim support clothing, and the like, since the material, apart from the specified drainage properties, for example may also effect an improved sweat absorption, an improvement of the blood supply to the muscle system and thus an improvement of the muscular performance. Moreover, the surface structure of the material and its supporting effect may also be perceived as being aesthetically pleasing.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings are shown:

FIG. 1 is a schematic lateral perspective view of a material of a first embodiment according to the invention;

FIG. 2 is a schematic lateral perspective view of a material of a second embodiment according to the invention;

FIG. 3 is a schematic lateral perspective view of a material of a third embodiment according to the invention;

FIG. 4 is a schematic lateral perspective view of a material of a fourth embodiment according to the invention;

FIG. 5 is a schematic lateral perspective view of a material of a fifth embodiment according to the invention;

FIG. 6 is a schematic lateral perspective view of a material of a sixth embodiment according to the invention;

FIG. 7 is a schematic lateral perspective view of a material of a seventh embodiment according to the invention;

FIG. 8 is a schematic lateral perspective view of a material of an eighth embodiment according to the invention;

FIG. 9 is a schematic lateral perspective view of a material of a ninth embodiment according to the invention;

FIG. 10 is a plan view of a material of a tenth embodiment according to the invention;

FIG. 11 is a plan view of a material of an eleventh embodiment according to the invention; and

FIG. 12 is a plan view of a material of a twelfth embodiment according to the invention.

### DETAILED DESCRIPTION OF THE INVENTION

The materials represented in the drawings in each case have a flat base body 2, wherein several protuberances 4, 4′, 4″, 4″', 4^{IV} and 4^{v} are deposited on a flat side. The base body 2 may selectively be formed by a textile, a plastic film or a foam body. The protuberances 4, 4′, 4″, 4″', 4^{v} and 4^{V} may also have a textile design, for example be stitched on the base body 2, or consist of plastic and be injected or bonded on the base body.

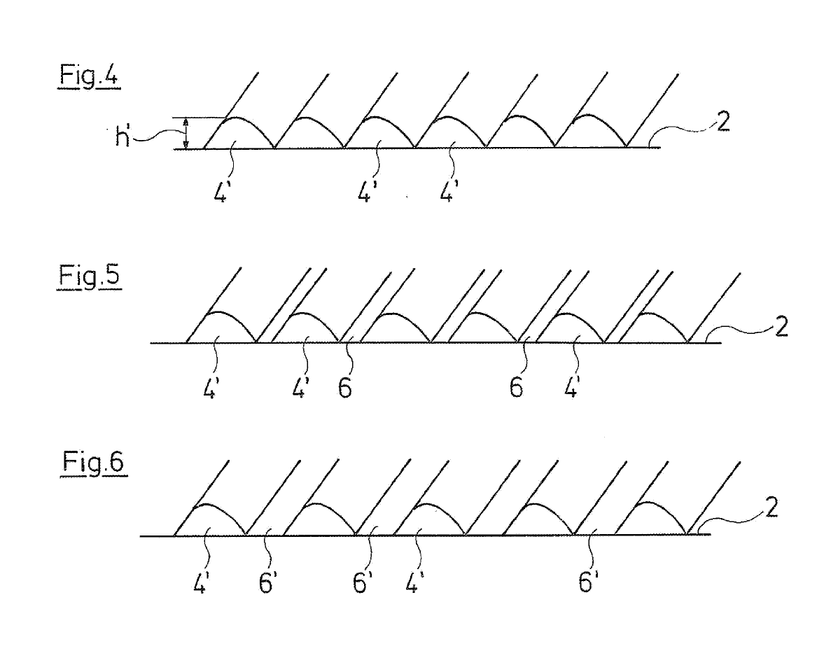

The protuberances 4 represented in the FIGS. 1 to 3, the protuberances 4′ represented in the FIGS. 4 to 6, and the protuberances 4″ represented in FIGS. 7 to 9 are in each case designed to be curved in a sinusoidal manner and differ merely with respect to their height h, h′ and h″. Thus, the height h″ of the protuberance 4″ is greater than the height h′ of the protuberance 4′, which in turn is greater than the height h of the protuberance 4. The height h, h′ or h″ is freely selectable and may be up to 30 mm.

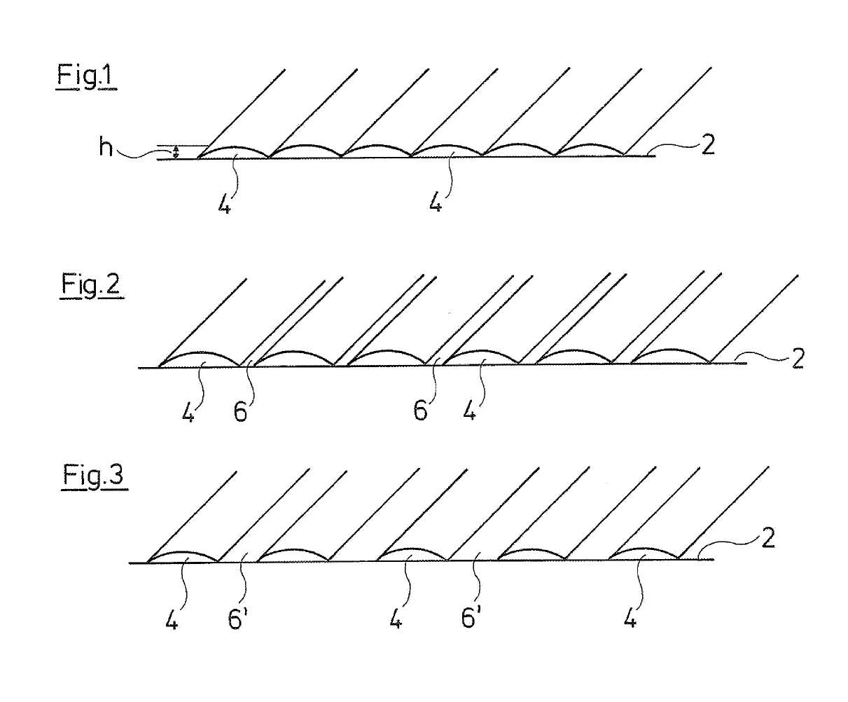

The materials represented in the FIGS. 1 to 3 differ merely to the extent that adjacent protuberances 4 are differently spaced in the individual Figures. Thus, the protuberances 4 in the embodiment represented in FIG. 1 are arranged directly adjacent one another, while the protuberances 4 in the embodiments represented in FIGS. 2 and 3 are spaced from one another. Thus, with the materials according to FIGS. 2 and 3, an intermediate space 6 or 6′ is formed between adjacent protuberances 4, whereby the application of the material, for example in combination with a bandage or a therapeutic piece of clothing, forms a channel for leading away tissue fluid. The width of the intermediate space 6′ in FIG. 3 is larger than that of the intermediate space 6 in FIG. 2 and may basically be infinitely selected up to a width of about 30 mm.

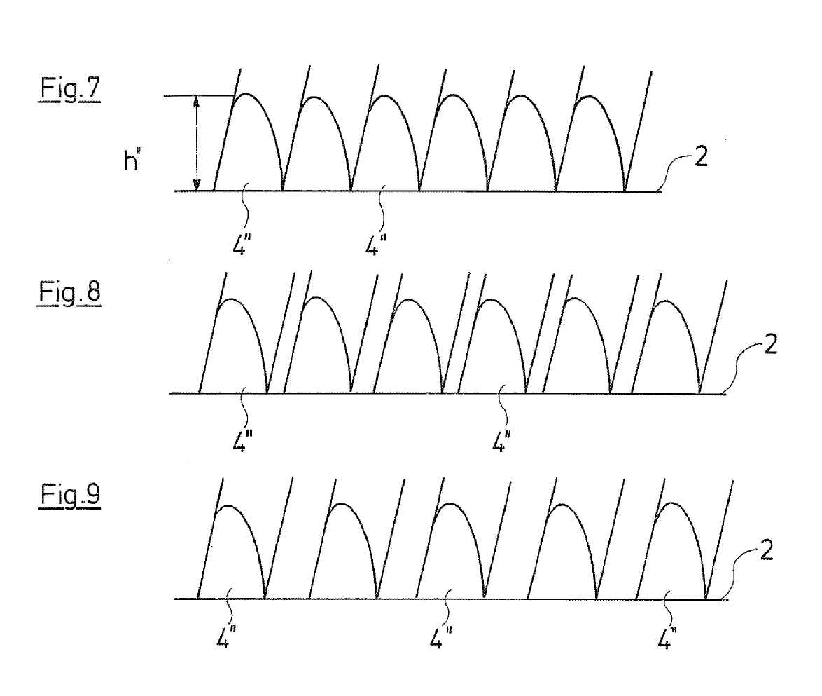

Analogously, the materials represented in the FIGS. 4 to 6 and the materials represented in FIGS. 7 to 9 differ by the spacing of adjacent protuberances 4′ and 4″, respectively. Thus, the protuberances 4′ in FIG. 4 and the protuberances 4″ in FIG. 7 are arranged directly adjacent one another, while an intermediate space 6 is formed between the protuberances 4′ in FIG. 5 and the protuberances 4″ in FIG. 8, and an intermediate space 6′ is formed between the protuberances 4′ in FIG. 6 and the protuberances 4″ in FIG. 9.

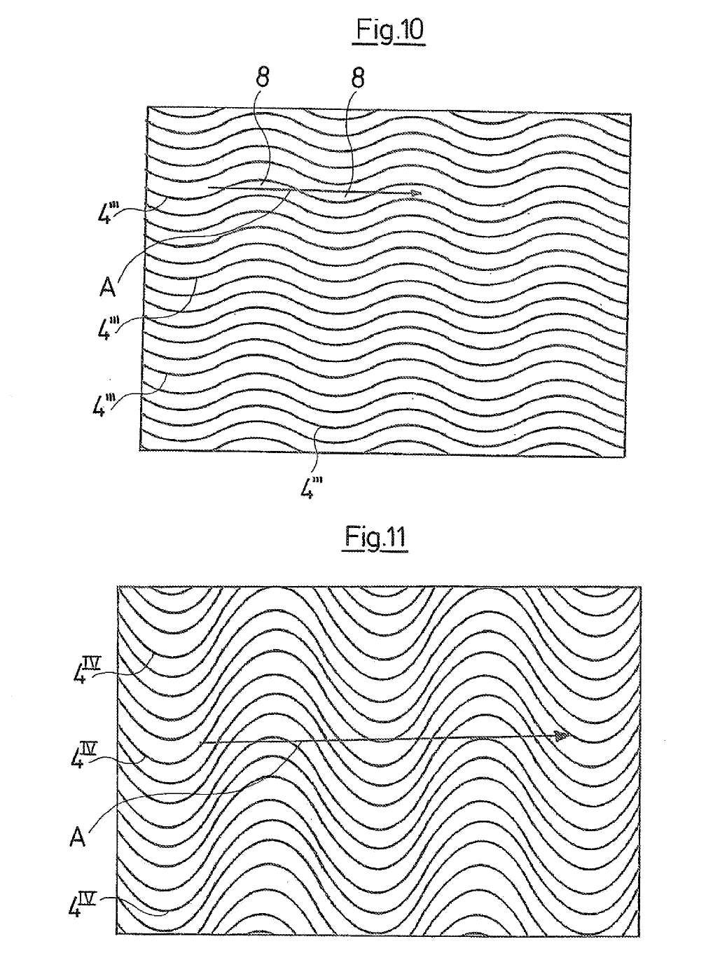

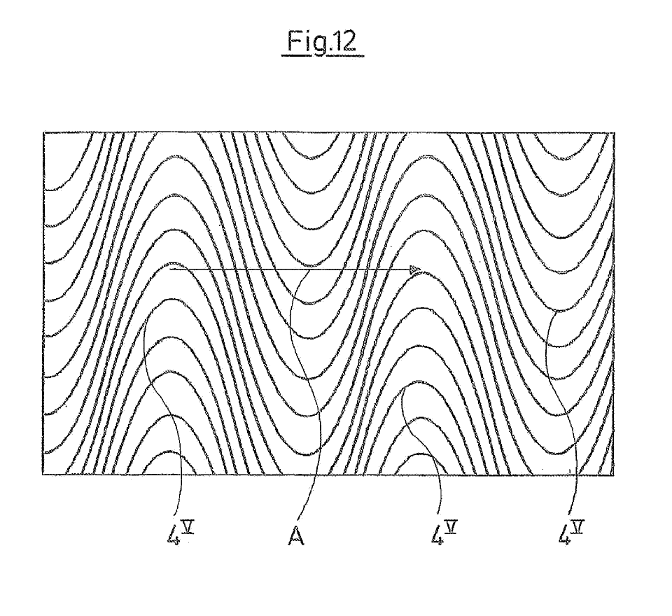

It is evident from FIGS. 10 to 12 that the protuberances 4″', 4^{IV} and 4^{V} which are provided there, although in each case being aligned in a basic direction A, do not run in this basic direction A in a straight-lined manner. Instead, the protuberances 4″', 4^{IV} and 4^{V} in the basic direction A have a meandering, which is to say serpentine course, so that for the most part they are aligned obliquely to the basic direction A. With respect to the basic direction A of the individual protuberances 4″', 4^{IV} and 4^{V}, the protuberances 4″', 4^{IV} and e form rounded curvatures 8 (see FIG. 10) which extend in the two opposite directions transversely to the basic direction A of the protuberances 4″', 4^{IV}, 4^{V}. The degree of curvature of these curvatures 8 is different in the embodiments in FIGS. 10 to 12 and may be freely selected in adaptation to the respective drainage tasks.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-15. (canceled)

16. A material for a product for compression of body parts, the material comprising a two-dimensional base body (2) and at least one linear protuberance (4, 4′, 4″,4″', 4^{IV}, 4^{V}) formed on at least one flat side of the base body and extending longitudinally in a basic direction (A), wherein the at least one protuberance (4, 4′, 4″, 4″', 4^{IV}, 4^{V}) comprises sections aligned obliquely and/or transversely to the basic direction (A).

17. The material according to claim 16, wherein the at least one protuberance (4, 4′, 4″',4_{IV}, 4^{V}) runs in a meandering manner in the basic direction (A).

18. The material according to claim 16, wherein a plurality of protuberances (4, 4′, 4″, 4″', 4^{IV}, 4^{V}) are formed on the base body (2), the protuberances lying adjacent one another in the basic direction (A).

19. The material according to claim 18, wherein the protuberances (4, 4′, 4″, 4″', 4^{IV}, 4^{V}) form a sinusoidal curvature (8) aligned normally to the at least one flat side of the base body (2).

20. The material according to claim 18, wherein the adjacent lying protuberances (4, 4′, 4″', 4_{IV}, 4_{V}) are spaced from one another.

21. . The material according to claim 18, wherein the adjacent lying protuberances (4, 4′, 4″, 4″', 4^{IV}, 4^{V}) border one another in an essentially direct manner.

22. The material according to claim 16, wherein the base body (2) is a textile.

23. The material according to claim 16, wherein the base body (2) is a film.

24. The material according to claim 16, wherein the base body (2) is foam body.

25. The material according to claim 16, wherein the at least one protuberance (4, 4′, 4″, 4″', 4^{IV}, 4^{V}) is deposited onto the base body (2).

26. The material according to claim 16, wherein the at least one protuberance (4, 4′, 4″, 4″', 4^{IV}, 4^{V}) forms a part of the base body (2).

27. A bandage comprising at least sections formed of the material according to claim 16.

28. The bandage according to claim 27, wherein the basic direction (A) of the at least one protuberance extends transversely to a longitudinal extension of the bandage.

29. A piece of clothing comprising at least sections formed of the material according to claim 16.

30. The piece of clothing according to claim 29, which comprises a stocking.

31. The piece of clothing according to claim 29, which comprises a bra.
